(19) United States
(12) Patent Application Publication
(10) Pub. No.: US 2005/0032680 A1
(43) Pub. Date: Feb. 10, 2005

(54) Method of treatment of vulval vestibulitis

(76) Inventor: Peter Knox, Workingham (GB)

Correspondence Address: KLAUBER & JACKSON, 411 HACKENSACK AVENUE, HACKENSACK, NJ, 07601, US

(21) Appl. No.: 10/494,224

(22) PCT Filed: Oct. 31, 2002
(86) PCT No.: PCT/GB02/04979

(30) Foreign Application Priority Data
Oct. 31, 2001 (GB) 0126253.4

(51) Int. Cl. A61K 38/13; A61K 31/4745

(52) U.S. Cl. 514/11; 514/291

(57) ABSTRACT

The invention relates to a method for the treatment of vulvar vestibulitis, by the topical application to an affected tissue of a therapeutically-effective amount of an agent that prevents or reduces the activation of T-lymphocyte cells, such as tacrolimus, everolimus, sirolimus or cyclosporin.

The invention relates to a method for the treatment of vulvar vestibulitis, by the topical application of an agent that prevents or reduces the activation of T lymphocytes.

Vulvar vestibulitis is a relatively common condition afflicting females, that can result in extreme discomfort and pain. It is a disorder that is often described in the general symptomatic category vulvodynia, which is a complex gynaecological syndrome. However careful clinical examination can enable a specific differential diagnosis of vulvar vestibulitis to be made. The condition can be acute and resolved in a few a months, or it can be chronic in nature. Although many different suggestions have been made, the aetiology of the condition is unclear.

Vulvar vestibulitis is associated with severe pain that is described as sharp, burning and a sense of rawness. Significant pain can result from the wearing of tight clothing and activities such as tampon insertion may have to be discontinued because of severe acute pain. In more severe cases, dyspareunia can totally preclude sexual intercourse. Thus, in addition to the local symptoms there are consequent psychosexual implications that can result in severely reduced quality of life.

A diagnosis of vulvar vestibulitis is made during clinical examination on a number of criteria:
exclusion of microbial infections such a candida and herpes;
pain on vestibular touch or attempted vaginal entry;
tenderness when a moist cotton Q-tip is gently applied to the vulvar vestibule;
erythema localized to the vulvar vestibule.

There can be in some cases variable amounts of itching, swelling and excoriation.

Due to the unknown aetiology, patients are currently treated with a wide range of therapeutic approaches, that range from psychological therapy, treatment with agents that have been developed for the relief of neurogenic pain, steroids, non-steroidal anti-inflammatories and even in some cases the surgical excision of the Bartholin's glands that are found within the vestibule. However, none of these approaches are entirely satisfactory.

There thus exists a great need for the identification of a method that is efficacious in the treatment of vulvar vestibulitis.

### SUMMARY OF THE INVENTION

According to the invention there is provided a method for the treatment of vulvar vestibulitis comprising the topical application to an affected tissue of a therapeutically-effective amount of an agent that prevents or reduces the activation of T-lymphocyte cells.

It has been discovered that the signs and symptoms of conditions such as vulvar vestibulitis arise from the infiltration and activation of populations of T-lymphocytes that migrate from systemic circulation into the vulvar tissue and the surrounding glands. Analysis of biopsies taken from vulvar vestibulitis lesions reveals that the tissue contains a cellular infiltrate containing T-lymphocytes. The method of the invention uses a pharmaceutical agent that prevents or reduces the activation of T-lymphocyte cells and thus inhibits the biological effects of T-cell activation.

When applied locally to an affected tissue, the agent reduces or removes T-cell function and thereby brings about clinical efficacy. The administration of such pharmaceuticals brings about a clinical response with an excellent risk-benefit profile because of the very low doses that are necessary when topical application is used, and because of the selectivity of the agents used in specific formulations.

By “vulvar vestibulitis” is meant a syndrome suffered by an identifiable subset of patients with vulvodynia (pain in the vulva), that is referred to by a number of different terms, including erythematous vulvitis en plaque, hyperasthesia of the vulva, minor vestibular adenitis, burning vulvar syndrome and focal vulvitis. A task force set up by the Tenth World Congress of the International Society for the Study of Vulvar Disease originally replaced the term “burning vulvar syndrome” with the term vulvodynia and defined the condition clinically. Vulvar vestibulitis is typically marked by a history of intermittent, then continuous discomfort in the vulva and introital dyspareunia. Examination reveals significant focal tenderness of the vestibule on contact or pressure in the absence of vulvar lesions or identifiable vulvovaginal infection. Macular erythematous patches may be noted along the hymeneal sulcus, at the openings of the major and minor vestibular glands. The paraurethral glands are also frequently involved. The erythema may vary in intensity and may not be apparent in all cases or at all times. Histology may reveal a chronic inflammatory cell reaction surrounding the vestibular glands.

A number of agents are known that prevent or reduce the activation of T-lymphocyte cells and that are thus suitable for use in the method of the invention. When a T-cell becomes activated there is a complex biochemical pathway initiated, offering a number of potential points of intervention.

For example, T cell activation results in the dephosphorylation of a protein present in the cytoplasm known as the nuclear factor of activated T-cells (NFAT). Following dephosphorylation, this translocates into the nucleus and there initiates the transcription of specific proteins including Interleukin-2 (IL-2). A number of potent inflammatory cascades are thereby initiated. The phosphatase that is responsible for the dephosphorylation of NFAT is called calcineurin. A number of pharmaceuticals such as cyclosporin and Tacrolimus prevent T-cell activation by blocking the activity of calcineurin. Interestingly, these and other agents first bind to a particular protein and the complex then binds to and inactivates calcineurin. For example, cyclosporin binds to a cytoplasmic protein that was initially referred to as cyclophilin. Later, it was discovered that cyclophilin is an enzyme with peptidyl prolyl cis-trans isomerase activity. Subsequently it was found that Tacrolimus and other macrolide compounds with similar activity bind to a different intracellular protein and now the general term “immunophilins” is used to describe all proteins that bind to immunosuppressive drugs such as cyclosporin, FK 506 and rapamycin.

Many agents that prevent or reduce the activation of T-lymphocyte cells have been successfully used previously in preventing organ rejection following transplant surgery and in certain other clinical conditions that involve gross immunological dysfunction. These agents exhibit a significant improvement over corticosteroids, although their use is nevertheless restricted by adverse side-effects. Used systemically, there is a risk of liver and kidney damage and always a risk of opportunistic infection. By applying these agents topically, these adverse side-effects may be circumvented.

Suitable agents for use in the method of the invention include, but are not limited to, cyclosporin, FK506 (Tacrolimus), everolimus and sirolimus, Imurek™, azathioprine sodium, brequinar sodium, spanidin™, gusperimus trihydrochloride (also known as deoxyspergualin), mizoribine (also known as bredinin), cellcept™. mycophenolate mofetil, neoral™, Cyclosporin A (also marketed as a different formulation of Cyclosporin A under the trademark Sandimmune™), prograf™. Tacrolimus (also known as FK-506) and rapimmune™, sirolimus (also known as rapamycin) and leflunomide (also known as HWA486). Cyclosporin, FK506 (Tacrolimus), everolimus and sirolimus are preferred agents for use in the method of the present invention. It will be appreciated that the selection of a suitable agent, to increase efficacy yet reduce the potential for toxic side-effects, will be well within the ability of the skilled reader. It is also likely that particular agents will exhibit different levels of efficacy and toxicity in different patients.

Preferably, the agent used in the method of the invention acts by binding to one or more immunophilins. By binding to an immunophilin, calcineurin may be inhibited.

Preferably, according to the invention, the agent may be topically applied to the patient in the form of a pharmaceutical composition containing the agent in a therapeutically-effective amount. The term “therapeutically effective amount” as used herein refers to an amount of agent that is needed to treat, ameliorate, or prevent vulvar vestibulitis, or to exhibit a detectable therapeutic or preventative effect. For any compound, the therapeutically effective dose will depend on the specific activity of the agent concerned and can be readily determined by routine experimentation, for example, by initial estimation either in cell culture assays, for example, of T cells from human peripheral blood that are activated in vitro, or in animal models, usually mice, rabbits, dogs, or pigs. The animal model may also be used to determine the appropriate concentration range and route of administration. Such information can then be used to determine useful doses in humans.

The precise effective amount for a human subject will depend upon the severity of the disease state, general health of the subject, age, diet, time and frequency of administration, drug combination(s), reaction sensitivities, and tolerance/response to therapy. This amount can be determined by routine experimentation and is within the judgement of the clinician. Compositions may be administered individually to a patient or may be administered in combination with other agents, drugs or hormones.

For example, agents that bind to immunophilins and inhibit calcineurin may be used in compositions that contain between around 0.01 and 1%, preferably, between 0.03 and 0.3%, more preferably around 0.1% w/v of active agent. For agents such as Tacrolimus, everolimus and sirolimus, a topically-applied composition for use in the invention is effective when containing between around 0.01 and 1%, preferably, between 0.03 and 0.3%, more preferably around 0.1%. A composition comprising cyclosporin as the active agent may contain between 0.01 and 1%, preferably, between 0.05 and 0.1% agent.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles and enable the pharmaceutical compositions to be formulated as liquids, gels, slurries, suspensions, and the like, for topical application to the patient.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, preservatives, detergents, colouring agents, and/or compounds with medicinal activity such as local anaesthetics or antiseptics and the like, may be present in such compositions.

Once formulated, the pharmaceutical compositions described above can be administered directly to the subject. The subjects to be treated may be animals, although the methods of the invention have particular application to human subjects.

By the term “topically-applied” is meant that the agent that prevents or reduces the activation of T-lymphocyte cells is applied to the skin locally at the point of inflammation in the area of the vulvar. Preferably the agent is applied topically to the vestibule.

Dosage treatment may be a single dose schedule or a multiple dose schedule. In one preferred regimen, the composition containing the agent that prevents or reduces the activation of T-lymphocyte cells may be applied daily, preferably to the vestibule. Similarly-effective alternatives to this regimen will be clear to those of skill in the art and include application of the agent one, two or three times each day, preferably to the vestibule.

According to a further aspect of the invention, there is provided the use of an agent that prevents or reduces the activation of T-lymphocyte cells in the manufacture of a medicament for the treatment of vulvar vestibulitis by topical application to an affected area. As described above, the medicament may be in the form of a pharmaceutical composition.

Various aspects and embodiments of the present invention will now be described in more detail by way of example. It will be appreciated that modification of detail may be made without departing from the scope of the invention.

### EXAMPLE

An ointment is prepared consisting of 0.1% Tacrolimus in a base of mineral oil, paraffin, propylene carbonate, white petroleum and white wax.

A small amount of the ointment, equivalent to a sphere of approximate diameter 2 mm should be applied to a finger after thoroughly washing hands. The ointment should be gently applied to the affected area and rubbed in completely. The hands should be washed again. No dressing should be applied and clothing around the area should be relatively loose.

The ointment should be applied twice a day, once in the morning and once in the evening, or otherwise as directed by a physician. Treatment can be continued for up to four weeks.

When the symptoms begin to reduce in severity, treatment should be continued for at least a week.

1. A method for the treatment of vulvar vestibulitis comprising the topical application to an affected tissue of a therapeutically-effective amount of an agent that prevents or reduces the activation of T-lymphocyte cells.

2. The method of claim 1 wherein the agent acts by binding to an immunophilin.

3. A method according to claim 2, wherein said binding to an immunophilin acts to inhibit calcineurin.

4. A method according to claim 1, wherein said agent is topically applied as a component of a pharmaceutical composition.

5. The method of claim 4, wherein the composition contains 0.03-0.3% w/v of a pharmaceutical known to reduce T-lymphocyte activity by binding to an immunophilin and inhibiting calcineurin.

6. The method of claim 5, wherein the agent is selected from the group consisting of FK506 (Tacrolimus), Everolimus and Sirolimus.

7. The method of claim 5, wherein the agent is applied as a composition containing 0.1% agent.

8. The method of claim 5, wherein said agent is cyclosporin.

9. The method of claim 8, wherein cyclosporin is applied as a composition containing between 0.05-0.1% agent.

10. The method of claim 1, wherein the pharmaceutical composition contains, in addition to the active agent, one or more compounds such as a buffer, a preservative, a detergent or a colouring agent, and/or a compound with medicinal activity, such as a local anaesthetic or antiseptic.

11. The method according to claim 1, wherein the agent is applied topically to the vestibule.

12. The method according to claim 1, wherein the agent is applied daily.

13. The method according to claim 1, wherein the agent is applied to the vestibule one, two or three times each day.

14. (canceled)

15. The method of claim 6, wherein the agent is applied as a composition containing 0.1% agent.
